(19) United States
(12) Patent Application Publication
(10) Pub. No.: US 2024/0398369 A1
(43) Pub. Date: Dec. 5, 2024

(54) IMAGING CONTROL APPARATUS, IMAGING CONTROL METHOD, AND RECORDING MEDIUM

(71) Applicant: Konica Minolta, Inc., Tokyo (JP)

(72) Inventor: Kojiro ONO, Narita-shi (JP)

(21) Appl. No.: 18/733,399

(22) Filed: Jun. 4, 2024

(30) Foreign Application Priority Data
Jun. 5, 2023 (JP) 2023-092262

(51) Int. Cl. A61B 6/00; A61B 6/40; A61B 6/46; G16H 10/60

(52) U.S. Cl. CPC A61B 6/544; A61B 6/4078; A61B 6/465; G16H 10/60

(57) ABSTRACT

An imaging control apparatus includes, a hardware processor that acquires information based on a number of times of simple X-ray imaging operations as information regarding a continuous imaging dose which is a dose in a series of X-ray imaging operations, wherein the hardware processor determines an imaging condition in the series of X-ray imaging operations in accordance with the acquired information based on the number of times of the simple X-ray imaging operations.

### BACKGROUND OF THE INVENTION

### Technical Field

The present invention relates to an imaging control apparatus, an imaging control method, and a recording medium.

### Description of Related Art

Conventionally, imaging with an appropriate amount of exposure has been required in simple X-ray imaging.

In relation to this, the following technology is disclosed in Japanese Unexamined Patent Publication No. 2001-305232, Japanese Unexamined Patent Publication No. 2002-253541. Specifically, it is a technique for controlling the dose of radiation emitted from a radiation generator in simple X-ray imaging to eliminate variations in image quality and exposure dose in simple X-ray imaging.

In dynamic imaging for obtaining a dynamic image including a plurality of frame images, imaging is also desirably performed with an appropriate amount of exposure, similarly to simple X-ray imaging.

In this connection, Japanese Unexamined Patent Publication No. 2017-136186 discloses a technique for controlling an irradiation dose of radiation to be constant each time dynamic imaging is performed.

Incidentally, in order to perform dynamic imaging with an appropriate amount of exposure, it is necessary to set imaging conditions for dynamic imaging so as to achieve the aforementioned amount of exposure. In a series of X-ray imaging such as dynamic imaging, there are much more factors to be considered for setting imaging conditions than in simple X-ray imaging Therefore, it is complicated and difficult to set imaging conditions for dynamic imaging.

Furthermore, dynamic imaging has only been used in the medical field for a short time Therefore, it is difficult to largely rely on the skill of a technician engaged in imaging as in simple X-ray imaging for setting imaging conditions in dynamic imaging. Therefore, it is extremely difficult to set imaging conditions so that the irradiation dose (exposure dose) in dynamic imaging and the quality of an image obtained by dynamic imaging become appropriate.

In addition, there is a problem in that there is no determination index for setting imaging conditions for realizing dynamic imaging with an appropriate exposure amount before the start of imaging. In the invention described in Japanese Unexamined Patent Publication No. 2017-136186, after the setting of the imaging condition is completed, control and image correction are performed so that imaging is performed in accordance with the setting. However, the invention described in Japanese Unexamined Patent Publication No. 2017-136186 does not disclose setting of imaging conditions before the start of imaging, and thus cannot solve the above-described problem.

### SUMMARY OF THE INVENTION

An object of the present invention, which has been made in view of the above-described problem, is to provide an imaging control apparatus, an imaging control method, and a non-transitory computer-readable recording medium storing a program, which are capable of controlling a dose in a series of X-ray imaging operations to an appropriate value.

To achieve at least one of the abovementioned objects, according to an aspect of the present invention, imaging control apparatus reflecting one aspect of the present invention includes: a hardware processor that acquires information based on a number of times of simple X-ray imaging operations as information regarding a continuous imaging dose which is a dose in a series of X-ray imaging operations, wherein the hardware processor determines an imaging condition in the series of X-ray imaging operations in accordance with the acquired information based on the number of times of the simple X-ray imaging operations.

To achieve at least one of the abovementioned objects, according to an aspect of the present invention, imaging control apparatus reflecting one aspect of the present invention includes: a hardware processor that acquires information based on a number of times of simple X-ray imaging operations as information regarding a continuous imaging dose which is a dose in a series of X-ray imaging operations, wherein the hardware processor presents an imaging condition in the series of X-ray imaging operations in accordance with the acquired information based on the number of times of the simple X-ray imaging operations.

To achieve at least one of the abovementioned objects, according to an aspect of the present invention, imaging control apparatus reflecting one aspect of the present invention includes: a hardware processor that acquires information based on a number of times of first simple X-ray imaging operations as information regarding a continuous imaging dose which is a dose in a series of X-ray imaging operations, wherein the hardware processor accepts an input of an imaging condition in the series of X-ray imaging operations, and wherein the hardware processor (i) outputs an alert based on information according to the acquired number of times of the first simple X-ray imaging operations and based on the accepted imaging conditions in the series of X-ray imaging operations or (ii) limits setting of the imaging condition or limits the series of X-ray imaging operations.

To achieve at least one of the abovementioned objects, according to an aspect of the present invention, an imaging control method reflecting one aspect of the present invention includes: acquiring information based on a number of times of simple X-ray imaging operations as information regarding a continuous imaging dose which is a dose in a series of X-ray imaging operations, wherein an imaging condition in the series of X-ray imaging operations is determined in accordance with the acquired information based on the number of times of the simple X-ray imaging operations.

To achieve at least one of the abovementioned objects, according to an aspect of the present invention, an imaging control method reflecting one aspect of the present invention includes: acquiring information based on a number of times of simple X-ray imaging operations as information regarding a continuous imaging dose which is a dose in a series of X-ray imaging operations, wherein an imaging condition in the series of X-ray imaging operations is determined in accordance with the acquired information based on the number of times of the simple X-ray imaging operations.

To achieve at least one of the abovementioned objects, according to an aspect of the present invention, an imaging control method reflecting one aspect of the present invention includes: acquiring information based on a number of times of simple X-ray imaging operations as information regarding a continuous imaging dose which is a dose in a series of X-ray imaging operations, accepting an input of an imaging condition in the series of X-ray imaging operations, and (i) outputting an alert based on the acquired information according to the number of times of the simple X-ray imaging operations and based on the accepted imaging condition in the series of X-ray imaging operations or (ii) limiting setting of the imaging condition or limiting the series of X-ray imaging operations.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a block diagram illustrating a radiation imaging system:

FIG. 2 is a block diagram illustrating an imaging control apparatus;

FIG. 3A is a diagram illustrating an example of an imaging condition table:

FIG. 3B is a diagram illustrating an example of an imaging condition and a reference ESD:

FIG. 3C is a diagram illustrating an example of an imaging condition and a reference ESD:

FIG. 4 is a flowchart illustrating a flow of an imaging condition determination processing:

FIG. 5 is a flowchart illustrating a flow of a number-of-times information acquisition processing;

FIG. 6 is a diagram illustrating an example of an imaging condition editing screen; and

FIG. 7 is a flowchart illustrating a flow of a during-imaging control processing.

### DETAILED DESCRIPTION

Hereinafter, one or more embodiments of the present invention will be described with reference to the drawings. However, the scope of the invention is not limited to the disclosed embodiments.

Hereinafter, embodiments of the present invention will be described with reference to the drawings. However, the technical scope of the present invention is not limited to the following embodiments and illustrated examples.

In the embodiment, dynamic imaging will be described as an example of a series of X-ray imaging, but the present invention is not limited thereto. The series of X-ray imaging includes fluoroscopic imaging, moving image imaging which is not dynamic imaging, tomosynthesis, imaging using dual energy subtraction, long length imaging, and the like. The long-length imaging is performed by emitting X-rays a plurality of times while moving an imaging apparatus.

### <1. Radiographic Imaging System>

The schematic configuration of a radiation imaging system (imaging system 100) according to the present embodiment will be described.

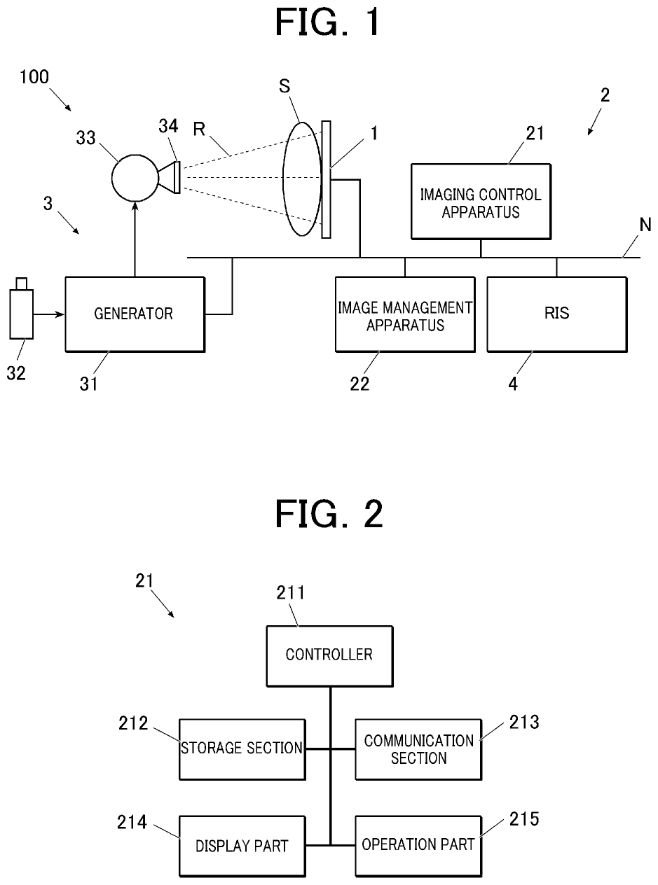

FIG. 1 is a block diagram illustrating the imaging system 100.

As illustrated in FIG. 1, the imaging system 100 includes a radiation image imaging apparatus (imaging apparatus 1), an image management system 2, a radiation generating apparatus (generating apparatus 3), and a radiology information system (RIS) 4.

The imaging apparatus 1, the image management system 2, the generating apparatus 3, and the RIS 4 can communicate with each other via, for example, a communication network N (a local area network (LAN), a wide area network (WAN), the Internet, or the like).

The imaging system 100 may be installed in an imaging mom or may be configured to be movable (for example, a medical cart).

The imaging system 100 may be equipped with an electronic medical record system or an ordering system instead of the RIS 4.

### [1-1. Radiation Generating Apparatus]

The generating apparatus 3 includes a generator 31, an irradiation instruction switch 32, a radiation source 33, and a dose detecting section 34.

The generating apparatus 3 applies the radiation R to an imaging site of a subject S located between the radiation source 33 of the generating apparatus 3 and the imaging apparatus 1 which are arranged opposite to each other with a gap therebetween.

The generator 31 applies a voltage corresponding to preset imaging conditions to the radiation source 33 (tube bulb) on the basis of operation of the irradiation instruction switch 32.

The imaging conditions include conditions related to the subject, such as imaging region, imaging direction, body position of the subject, physique of the subject, state of the subject, age of the subject, and sex of the subject, for example.

The imaging region is, for example, a chest, a leg, or the like.

The physique of the subject may be designated by a numerical value such as weight, height, body mass index (BMI), or body thickness of the subject, or may be a range-based classification. The range-based classifications are, for example, large/medium/small, thin/average/thick, and the like.

The state of the subject includes type of breathing such as breath holding and deep breathing.

The age of the subject may be designated by a numerical value or may be a range-based classification (child, adult, or the like).

The sex of the subject may be identified from physical structural characteristics.

The imaging conditions may also include, for example, conditions related to irradiation with radiation R, such as a type of examination, tube voltage and tube current, imaging time, current-time product (mAs value), distance (focus-film distance (FFD) or source image receptor distance (SID)) from the radiation source 33 to the imaging apparatus 1, frame rate, the presence or absence of a grid, the type of additional filter, and the manufacturer and/or classification of the generating apparatus 3. The frame rate is the number of frames acquired per second.

When a voltage is applied from the generator 31, the radiation source 33 generates radiation R (e.g., X-rays) in a dose corresponding to the applied voltage.

The dose detecting section 34 is located on the side of the radiation source 33 to which the radiation R is applied, and detects the dose of the radiation R applied from the radiation source 33.

The generating apparatus 3 outputs the dose of the radiation R detected by the dose detecting section 34 to the image management system 2.

In addition, the generating apparatus 3 generates the radiation R in a mode corresponding to the form (a still image or a dynamic image having a plurality of frame images) of the radiation image to be generated.

In the case of a still image, the generating apparatus 3 performs emission of the radiation R only once for each pressing of the irradiation instruction switch 32.

In the case of the dynamic image, the generating apparatus 3 repeats the emission of the pulsed radiation R a plurality of times for a predetermined amount of time (for example, 15 times per second) for each pressing of the irradiation instruction switch 32. Alternatively, in the case of a dynamic image, the generating apparatus 3 continues the emission of the radiation R for a predetermined amount of time for each depression of the irradiation instruction switch 32.

### [1-2. Radiation Image Imaging Apparatus]

The imaging apparatus 1 generates digital data of a radiation image in which the imaging region of the subject S is imaged.

The imaging apparatus 1 is, for example, a portable flat panel detector (FPD) apparatus.

Specifically, although not illustrated in the drawings, the imaging apparatus 1 includes a sensor substrate, a scanner, a reader, a controller, a communication section, and the like.

On the sensor substrate, imaging elements that generate electric charges corresponding to the dose in response to receiving the radiation R and switch elements that accumulate and release the electric charges are arranged two dimensionally (in a matrix).

The scanner switches each switch element on/off.

The reader reads the quantity of electric charge released from each pixel as a signal value.

The controller controls each section and generates a radiation image from the plurality of signal values read by the reader.

The communication section transmits data of the generated radiographic image, various signals, and the like to an external apparatus (image management system 2, generating apparatus 3, or the like), and receives various information and various signals from the external apparatus.

The imaging apparatus 1 accumulates and releases charges and reads signal values in synchronization with the timing at which the radiation R is emitted from the generating apparatus 3. Accordingly, the imaging apparatus 1 generates still image data, which is image data of a still image, or dynamic image data, which is image data of a dynamic image.

When generating still image data, the imaging apparatus 1 generates a radiation image only once per pressing of the irradiation instruction switch 32.

In the case of generating the dynamic image data, the imaging apparatus 1 repeatedly generates frame images forming the dynamic image a plurality of times per predetermined amount of time for each pressing of the irradiation instruction switch 32. The plurality of times is, for example, 15 times per second.

The imaging apparatus 1 may be integrated with the generating apparatus 3 (e.g., a computed tomography (CT) apparatus or the like).

Furthermore, the imaging apparatus 1 may cause a display apparatus connected to itself to display the generated dynamic image in real time.

### [1-3. Image Management System]

The image management system 2 includes an imaging control apparatus 21 and an image management apparatus 22.

### (1-3-1. Imaging Control Apparatus)

The imaging control apparatus 21 receives the still image data and/or the dynamic image data from the imaging apparatus 1.

The imaging control apparatus 21 is constituted by a PC, a dedicated device, or the like, and also serves as a console. That is, the imaging control apparatus 21 sets various imaging conditions in at least one of the imaging apparatus 1 and the generating apparatus 3. The various imaging conditions include a tube voltage, a tube current, a current-time product (mAs value), an imaging region, an imaging direction, a frame rate, and an imaging time.

The imaging control apparatus 21 sets the imaging conditions on the basis of imaging order information acquired from another system (RIS 4, or the like) or an operation performed by a user (such as a technician, for example).

The imaging control apparatus 21 may be separate from the console.

The imaging control apparatus 21 may also serve as another device other than the console.

FIG. 2 is a block diagram illustrating the imaging control apparatus 21.

As illustrated in FIG. 2, the imaging control apparatus 21 includes a controller 211, a storage section 212, a communication section 213, a display part 214, and an operation part 215.

The controller 211, the storage section 212, the communication section 213, the display part 214, and the operation part 215 are electrically connected to each other via a bus or the like.

The controller 211 includes a central processing unit (CPU), a random access memory (RAM), a read only memory (ROM), and the like.

The ROM stores various programs to be executed by the CPU, parameters necessary for executing the programs, and the like.

The CPU reads out various programs stored in the ROM, deploys the programs into the RAM, executes various processes in accordance with the deployed programs, and centrally controls operations by each component of the imaging control apparatus 21.

The storage section 212 includes a nonvolatile memory, a hard disk, and the like.

The storage section 212 stores a reference entrance surface dose (ESD), which is the entrance surface dose per simple X-ray imaging operation. The entrance surface dose is equivalent to the air dose, including scattered rays from the surface of the subject.

The reference ESD is, for example, a standard ESD (standard dose) in simple X-ray imaging used in a facility where dynamic imaging is performed.

The reference ESD has, for example, a value corresponding to a subject. Specifically, the reference ESD is a value corresponding to, for example, the physique of the subject, the age of the subject, the sex of the subject, or the like.

The reference ESD may be a diagnostic reference level for general imaging (simple X-ray imaging). As the diagnostic reference level, for example, “National Diagnostic Reference Levels in Japan (2020 edition) Japan DRLs, Jul. 3, 2020” is used in Japan. In the United States, for example, “ACR-AAPM-SPR PRACTICE PARAMETER FOR DIAGNOSTIC REFERENCE LEVELS AND ACHIEVABLE DOSES IN MEDICAL X-RAY IMAGING Revised 2018 (Resolution 40)” is used. In Europe, for example, “Lung scan dose” on page 24 of “Radiation Protection 109” is used.

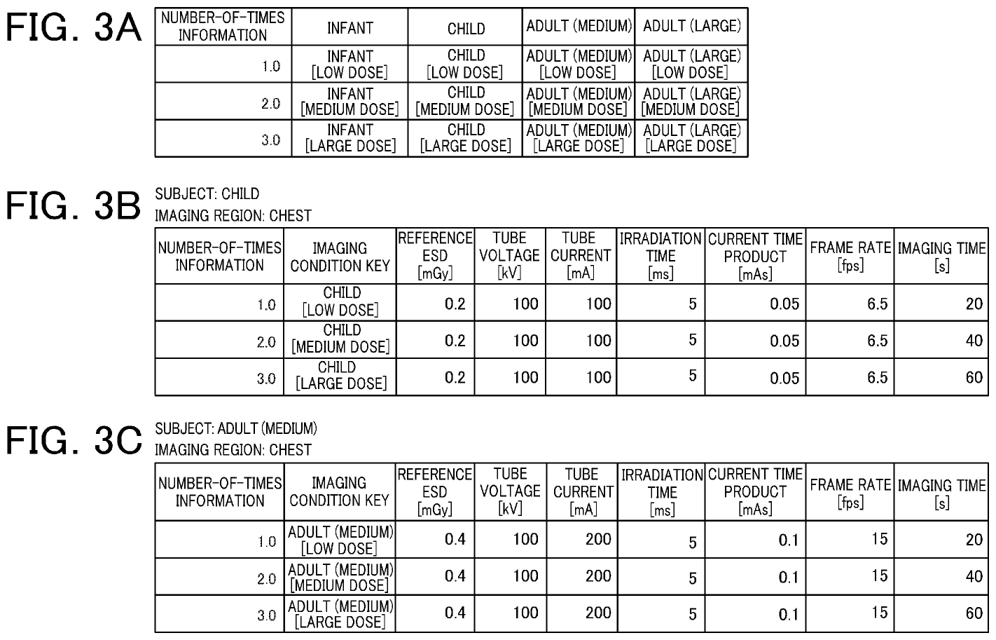

The storage section 212 stores the imaging condition table shown in FIG. 3A.

In the example shown in FIG. 3A, the imaging condition table has 12 kinds of imaging condition keys corresponding to the ages or physiques of the subjects for each number-of-times information described later. The imaging condition and the reference ESD are set for each of the imaging condition keys.

FIG. 3B illustrates an example of the imaging condition and the reference ESD for each imaging condition key in a case where the subject is a child and the imaging region is the chest. FIG. 3C illustrates an example of the imaging condition and the reference ESD for each imaging condition key in a case where the subject is an adult, the physique is at a middle level, and the imaging region is the chest. In FIG. 3B and FIG. 3C, a tube voltage, a tube current, an irradiation time, a current-time product, a frame rate, and an imaging time are set as imaging conditions.

The values of the tube voltage, the tube current, the irradiation time, the current-time product, the frame rate, and the imaging time as the imaging conditions are not limited to the examples illustrated in FIG. 3B and FIG. 3C.

In the examples shown in FIG. 3B and FIG. 3C, the imaging condition corresponding to the respective number-of-times information is set by differentiating the amount of imaging time, but the present invention is not limited thereto. Any one of the tube voltage, the tube current, the irradiation time, the current-time product, and the frame rate may be vaned to set the imaging conditions corresponding to the respective number-of-times information. Alternatively, by changing two or more items of the tube voltage, the tube current, the irradiation time, the current-time product, the frame rate, and the imaging time, the imaging conditions corresponding to the respective number-of-times information may be set.

The communication section 213 includes a communication module and the like.

The communication section 213 transmits and receives various signals and various data to and from an external apparatus (the imaging apparatus 1, the image management apparatus 22, the generating apparatus 3, the RIS 4, or the like) connected in a wired or wireless manner via the communication network N.

The display part 214 includes, for example, a liquid crystal display (LCD), an electronic luminescent display (ELD), a cathode ray tube (CRT), or the like.

The display part 214 displays a radiation image or the like corresponding to an image signal received from the controller 211.

The operation part 215 includes a keyboard (cursor keys, number input keys, various function keys, and the like), a pointing device (mouse or the like), a touch screen layered on the surface of the display part 214, and the like.

The operation part 215 outputs, to the controller 211, a control signal responsive to an operation performed by the user.

The imaging control apparatus 21 may not include the operation part 215, and may receive a control signal from an input apparatus provided separately from the imaging control apparatus 21 via the communication section 213 or the like, for example. The imaging control apparatus 21 may not include the display part 214 and may output the image signal to a display apparatus (monitor) provided separately from the imaging control apparatus 21.

When the external apparatus (the image management apparatus 22 or the like) includes an operation part, the imaging control apparatus 21 may receive a control signal from the operation part of the external apparatus. When the external device includes a display part, the imaging control apparatus 21 may output an image signal to the display part of the external apparatus. In other words, the imaging control apparatus 21 may share the display part and the operation part with the external apparatus.

### (1-3-2. Image Management Apparatus)

The image management apparatus 22 manages image data (still image data, dynamic image data, and the like) generated by the imaging apparatus 1.

The image management apparatus 22 is a picture archiving and communication system (hereinafter, PACS), an image diagnosis workstation (hereinafter, Imaging Work Station or IWS), or the like.

The image management apparatus 22 may be separate from the PACS or the IWS.

The image management apparatus 22 may also serve as an apparatus other than the PACS or the IWS.

The image management apparatus 22 stores the image data (still image data, dynamic image data, and the like) generated by the imaging apparatus 1. The image management apparatus 22 may be capable of storing image data other than the image data generated by the imaging apparatus 1.

The image management apparatus 22 stores radiation dose information corresponding to a radiation dose in imaging for obtaining the still image data, the dynamic image data, and the like received from the imaging control apparatus 21.

### <2. Operation by Radiation Image Imaging System>

The imaging system 100 according to the present embodiment operates as follows.

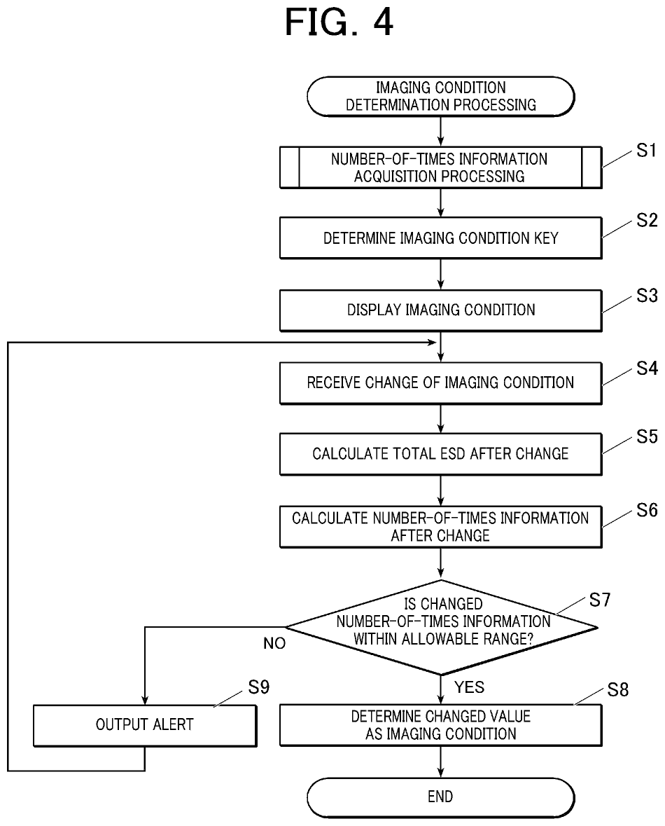

The controller 211 of the imaging control apparatus 21 executes an imaging condition determination process illustrated in FIG. 4 before the dynamic imaging is performed m the generating apparatus 3 and the imaging apparatus 1.

### (Imaging Condition Determination Processing)

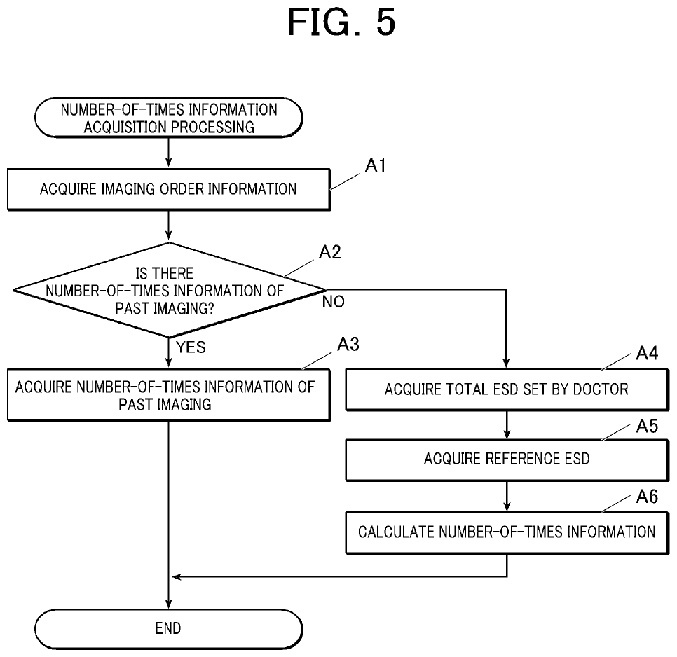

The controller 211 of the imaging control apparatus 21 executes the number-of-times information acquisition processing illustrated in FIG. 5 (step S1).

### (Number-of-Times Information Acquisition Processing)

The controller 211 acquires imaging order information from the RIS 4 (step A1).

Imaging order information is information in which information necessary for performing imaging in the generating apparatus 3 and the imaging apparatus 1 is set.

The imaging order information includes conditions related to the subject, information on the incident surface dose in a series of dynamic imaging operations set by the doctor, and the like. Hereinafter, the incident surface dose in the series of dynamic imaging processes set by the doctor is also referred to as the total ESD set by the doctor.

The series of dynamic imaging is imaging from issuance of an instruction to start dynamic imaging (e.g., pressing of the irradiation instruction switch 32) to release of the imaging instruction (e.g., release of the pressing of the irradiation instruction switch 32).

For example, the conditions related to the subject are the physique of the subject, the age of the subject, the sex of the subject, and the like.

The imaging order information may include information corresponding to the number of times of dynamic imaging of the subject in the past. The information corresponding to the number of times of past dynamic imaging of the subject is information corresponding to the dose of radiation emitted to the subject at the time of past dynamic imaging of the subject.

The number-of-times information is information indicating how many times the dose in a series of dynamic imaging corresponds to the dose per simple X-ray imaging. That is, the number-of-times information is calculated by the following expression.

Number-of-times information=incident surface dose in a series of dynamic imaging (total ESD)/incident surface dose per simple X-ray imaging (reference ESD).

The number-of-times information is information based on the number of times of simple X-ray imaging.

Next, the controller 211 determines whether the imaging order information acquired in step A1 includes information on the number of times of past dynamic imaging of the subject (step A2).

The case where the imaging order information includes information on the number of times of past dynamic imaging of the subject (step A2; YES) will be described. In this case, the controller 211 acquires, from the imaging order information, information corresponding to the number of times of past dynamic imaging of the subject (step A3), and ends the present processing.

On the other hand, a case where the imaging order information does not include information on the number of times of imaging of the subject in the past dynamic imaging (step A2; NO) will be described. In this case, the controller 211 acquires the total ESD set by the doctor from the imaging order information (step A4).

Next, the controller 211 acquires, from the storage section 212, a reference ESD corresponding to the condition related to the subject (step A5).

To be specific, the controller 211 acquires the reference ESD corresponding to the condition related to the subject included in the imaging order information acquired m step A1.

The controller 211 may acquire, as the reference ESD, a diagnostic reference level corresponding to a condition related to the subject.

Next, the controller 211 calculates number-of-times information on the basis of the total ESD and the reference ESD (step A6), and ends the present processing. The total ESD is the total ESD set by the doctor that the controller 211 acquired in step A4. The reference ESD is a reference ESD corresponding to the condition related to the subject acquired by the controller 211 in step A5.

Instead of executing the number-of-times information acquisition processing, the controller 211 may acquire the number-of-times information by accepting an input operation of the number-of-tunes information by the user via the operation part 215.

The imaging order information transmitted from RIS 4 may include number-of-times information. In this case, the controller 211 acquires the number-of-times information from the imaging order information.

That is, the controller 211 acquires number-of-times information (information based on the number of times of simple X-ray imaging) from the RIS 4 (radiology information system).

As described above, the controller 211 acquires the number-of-times information.

That is, the controller 211 acquires number-of-times information (information based on the number of times of simple X-ray imaging) as information about a continuous imaging dose, which is a dose in a series of X-ray imaging. The controller 211 functions as an acquirer. Step S1 is an acquiring step.

The information based on the number of simple X-ray imaging operations is the number-of-times information acquired by the controller 211 in step S1.

The information based on the number of times of simple X-ray imaging is information corresponding to at least one of the age of the subject, the sex of the subject, the physique of the subject, the dose in past X-ray imaging of the subject, and a standard dose.

Referring back to FIG. 4, the controller 211 determines an imaging condition key on the basis of the number-of-times information and the conditions related to the subject acquired in step S1 (step S2).

To be specific, the controller 211 determines, from the imaging condition table stored in the storage section 212, an imaging condition key corresponding to the number-of-times information and the age or physique of the subject acquired in step S1.

That is, the controller 211 determines an imaging condition key for which imaging conditions are set, according to the number-of-times information (information based on the number of times of simple X-ray imaging) acquired as the acquirer That is, the controller 211 determines imaging conditions in a series of X-ray imaging operations in accordance with the number-of-times information (information based on the number of times of simple X-ray imaging) acquired as the acquirer. The controller 211 functions as a determiner Step S2 is a determining step.

The imaging conditions in the series of X-ray imaging are conditions corresponding to the subject.

The imaging conditions in the series of X-ray imaging are at least one of a tube voltage, a tube current, a frame rate, and an imaging time.

Next, the controller 211 displays the imaging condition set to the imaging condition key determined in step S2 on the display part 214 (step S3).

That is, the controller 211 presents imaging conditions in a series of X-ray imaging operations in accordance with the number-of-times information (information based on the number of times of simple X-ray imaging) acquired as an acquirer. The controller 211 functions as a presenter. Step S3 is a presenting step.

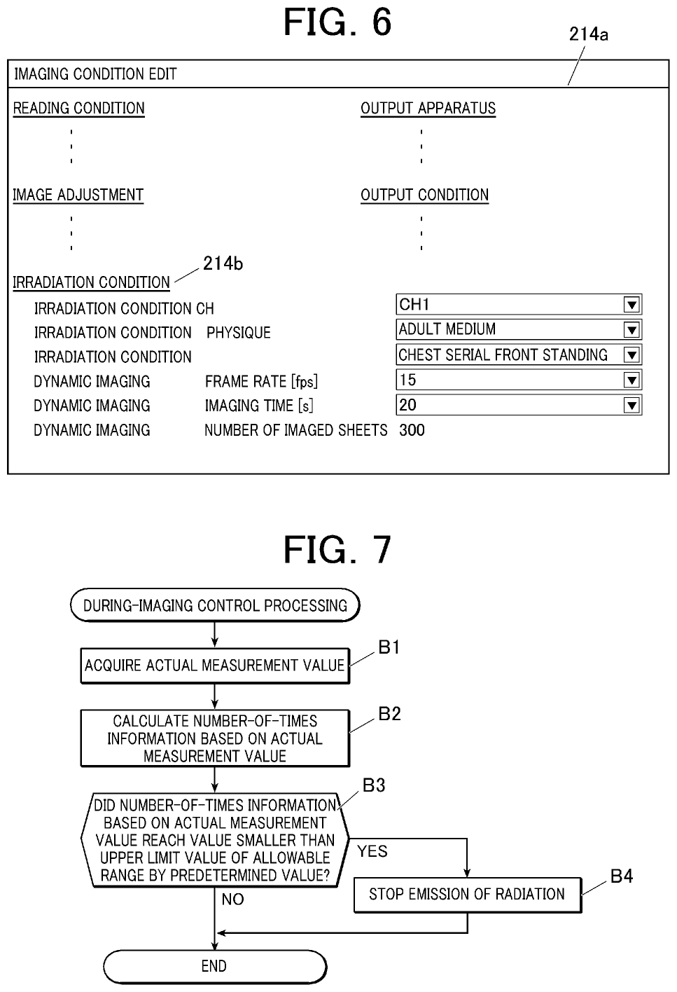

FIG. 6 illustrates an imaging condition editing screen 214a that the controller 211 displays on the display part 214.

In the example illustrated in FIG. 6, in step S3, the controller 211 displays a frame rate and an imaging time as the imaging conditions in an irradiation condition field 214b.

In step S3, the controller 211 may display the tube voltage, the current-time product, and the like, which are the imaging conditions, in the irradiation condition field 214b.

Next, the controller 211 accepts a change made by the user to the imaging conditions displayed in step S3 via the operation part 215 (step S4).

That is, the controller 211 accepts input of imaging conditions in a series of X-ray imaging operations. The controller 211 functions as an acceptor. The step S4 is an accepting step.

In the example illustrated in FIG. 6, in step S4, the user changes the frame rate and/or the imaging time displayed in the irradiation condition field 214b.

In step S4, the user may change the tube voltage, the current-time product, and the like, which are imaging conditions.

When the change of the imaging condition by the user is accepted, the controller 211 calculates the total ESD based on the changed imaging condition (step S5).

Next, the controller 211 calculates number-of-times information after the change based on the total ESD after the change calculated in step S5 and the reference ESD acquired in step A5 (step S6).

A case where, in the number-of-times information acquisition processing, the imaging order information included number-of-times information obtained when the subject was subjected to dynamic imaging in the past (step A2; YES) will be described. In this case, in step S6, the controller 211 acquires, from the storage section 212, a reference ESD corresponding to the condition related to the subject. Next, the controller 211 calculates number-of-times information after the change based on the total ESD after the change calculated in step S5 and the acquired reference ESD.

Next, the controller 211 determines whether the changed number-of-times information calculated in step S6 is within an allowable range (step S7).

The upper limit value in the allowable range of the number-of-times information is the number-of-times information acquired in step S1.

The lower limit value in the allowable range of the number-of-times information is a value at which the image quality of the image generated under the changed imaging condition is equal to or higher than a predetermined threshold value, and is set in advance.

If the changed number-of-times information is within the allowable range (step S7; YES), the controller 211 determines the value for which the change has been accepted in step S4 as the final imaging condition (step S8). The controller 211 ends the present processing.

In a case where the changed number-of-times information is not within the allowable range (step S7; NO), the controller 211 displays an alert on the display part 214 (step S9), and the process proceeds to step S4.

That is, the controller 211 outputs an alert based on the number-of-times information acquired in step S1 and the imaging conditions in the series of X-ray imaging operations accepted as the acceptor. The number-of-times information acquired in step S1 is information based on the number of times of the first simple X-ray imaging acquired as the acquirer. The controller 211 functions as an alert outputter. Step S9 is an alert outputting step.

Specifically, the controller 211 serving as an alert outputter outputs an alert when the information based on the number of times of the second simple X-ray imaging exceeds the information based on the number of times of the first simple X-ray imaging. The information based on the number of times of the second simple X-ray imaging is information based on the number of times of simple X-ray imaging based on the imaging conditions in the series of X-ray imaging accepted as the acceptor.

In step S4, the controller 211 accepts a change in the imaging conditions from the user again.

That is, the controller 211 restricts the setting of the imaging conditions based on the number-of-times information acquired in step S1 and the imaging conditions in the series of X-ray imaging operations accepted as the acceptor. The number-of-times information acquired in step S1 is information based on the number of times of the first simple X-ray imaging acquired as the acquirer. The controller 211 functions as a limiter. This step is the limiting step.

Specifically, the controller 211 as the limiter limits setting of the imaging conditions in a case where the information based on the number of times of the second simple X-ray imaging exceeds the information based on the number of times of the first simple X-ray imaging. The information based on the number of times of the second simple X-ray imaging is information based on the number of times of simple X-ray imaging based on the imaging conditions in the series of X-ray imaging accepted as the acceptor.

The controller 211 sets the imaging conditions determined in the image capturing condition determination processing to the imaging apparatus 1 and the generating apparatus 3.

The imaging apparatus 1 and the generating apparatus 3 perform imaging based on the set imaging conditions.

The imaging condition table includes 12 types of imaging condition keys in the example illustrated in FIG. 3A, but is not limited thereto. The imaging condition table may have, for example, three kinds of imaging condition keys for each number-of-times information. For example, the imaging condition table has imaging condition keys of “low dose”, “medium dose”, and “high dose”.

In this case, in step S2, the controller 211 determines the imaging condition key based on the number-of-times information acquired in step S1.

Next, in step S3, the controller 211 changes the imaging condition set in the imaging condition key determined in step S2 to a value corresponding to the condition relating to the subject acquired in step S1. The condition relating to the subject is the age or physique of the subject. Next, the controller 211 displays, on the display part 214, the imaging condition changed in accordance with the condition regarding the object.

The controller 211 may execute steps S5 to S9 of the imaging condition determination processing at the timing when the irradiation instruction switch 32 is pressed by the user to start the dynamic imaging. In this case, in step S9, the controller 211 may control the generating apparatus 3 so that the radiation R is not emitted.

The controller 211 may control so that the irradiation instruction switch 32 of the generating apparatus 3 cannot be pressed instead of step S9 of the imaging condition determination processing.

The controller 211 may control each apparatus of the imaging system 100 so that imaging cannot be performed instead of step S9 of the imaging condition determination processing.

That is, the controller 211 as a limiter limits the series of X-ray imaging on the basis of the number-of-times information acquired in step S1 and the imaging conditions in the series of X-ray imaging operations accepted by the acceptor. The number-of-times information acquired in step S1 is information based on the number of times of the first simple X-ray imaging acquired as the acquirer. This step is the limiting step.

Specifically, the controller 211 as the limiter limits the series of X-ray imaging in a case where the information based on the number of times of the second simple X-ray imaging exceeds the information based on the number of times of the first simple X-ray imaging. The information based on the number of times of the second simple X-ray imaging is information based on the number of times of simple X-ray imaging based on the imaging conditions in the series of X-ray imaging accepted as the acceptor.

Next, during-imaging control processing that the controller 211 executes during imaging by the imaging apparatus 1 and the generating apparatus 3 will be described.

FIG. 7 shows a flowchart of the during-imaging control processing.

### (During-Imaging Control Processing)

The controller 211 acquires the actual measurement value which is the dose of the radiation R detected by the dose detecting section 34 from the generating apparatus 3 (step B1).

That is, the controller 211 acquires the dose of radiation emitted to the object. The controller 211 functions as a dose acquirer.

Next, based on the actual measurement value acquired in step B1 and the reference ESD acquired in step A5, the controller 211 calculates number-of-times information based on the actual measurement value (step B2).

A case where, in the number-of-times information acquisition processing, the imaging order information included number-of-times information obtained when the subject was subjected to dynamic imaging in the past (step A2; YES) will be described. In this case, in step B2, the controller 211 acquires, from the storage section 212, a reference ESD corresponding to the condition related to the subject. Next, based on the actual measurement value acquired in step B1 and the acquired reference ESD, the controller 211 calculates number-of-times information based on the actual measurement value.

Next, the controller 211 determines whether the number-of-times information based on the actual measurement value calculated in step B2 has reached a value smaller than the upper limit value of the allowable range by a predetermined value (step B3). The upper limit value of the allowable range is the number-of-times information acquired in the imaging condition determination processing step S1. The predetermined value is set in advance.

In a case where the number-of-times information based on the actual measurement value does not reach the value smaller than the upper limit value of the allowable range by the predetermined value (step 33; NO), the controller 211 ends the present process.

A case where the number-of-times information based on the actual measurement value has reached a value smaller than the upper limit value of the allowable range by a predetermined value (step B3; YES) will be described. In this case, the controller 211 controls the generating apparatus 3 to stop the emission of the radiation R, and ends the process. And/or, in this case, the controller 211 may display an alert on the display part 214.

That is, the controller 211 stops the radiation irradiation when the number-of-times information based on the actual measurement value exceeds a value smaller than the information based on the number of times of the first simple X-ray imaging by a predetermined value.

The number-of-times information based on the actually measured value is information based on the number of times of the third simple X-ray imaging that is based on the dose of radiation emitted to the subject acquired by the dose acquirer.

The controller 211 as an alert outputter outputs an alert when the number-of-times information based on the actual measurement value exceeds a value that is smaller by a predetermined value than the information based on the number of times of the first simple X-ray imaging.

### (Others)

During imaging by the imaging apparatus 1 and the generating apparatus 3, the controller 211 displays, on the display part 214, the remaining time of the imaging time included in the imaging conditions determined in the imaging condition determination processing.

Alternatively, during imaging by the imaging apparatus 1 and the generating apparatus 3, the controller 211 may notify the user of the remaining time of the imaging time included in the imaging conditions determined in the imaging condition determination processing by outputting a sound or the like. In this case, the controller 211 may count down the remaining time. Thus, even in a case where a technician who performs imaging cannot visually recognize the display part 214 during imaging, the technician can grasp the remaining time of the imaging time.

A case in which the radiation R is emitted beyond the imaging time included in the imaging conditions determined in the imaging condition determination process during imaging by the imaging apparatus 1 and the generating apparatus 3 will be described. In this case, the controller 211 controls the generating apparatus 3 to stop the irradiation of the radiation R. And/or, in this case, the controller 211 may display a dose over alert on the display part 214. Alternatively, in this case, the controller 211 may notify the user of the dose over alert by outputting sound, light, or the like.

The controller 211 may make the display form of the dose over alert, the type of sound of voice, the color of light, and the like different from those of other imaging abnormality alerts.

In a case where an imaging abnormality (another imaging abnormality) other than the case where the radiation R is emitted beyond the imaging time occurs, it is necessary to urgently stop the imaging. Therefore, an alert for another imaging abnormality may be notified not only to a technician but also to a patient who is a subject. However, in a case in which the radiation R is emitted beyond the imaging time, the imaging may be continued depending on the situation. Therefore, the controller 211 may be able to notify only the technician of the dose over alert so that the patient does not feel uneasy about the fact that the imaging is continued although the dose over alert is notified. For example, the controller 211 provides notification of the dose over alert exclusively to the outside of the imaging chamber. Alternatively, the controller 211 notifies only the imaging control apparatus 21 (console) of the dose over alert. Alternatively, the controller 211 does not notify the dose over alert with a PDA (Personal Digital Assistance) attached to the imaging room. Alternatively, the controller 211 does not notify of the dose over alert by the medical cart.

### 3. Effects

As described above, the imaging control apparatus 21 according to the present embodiment includes an acquirer (controller 211) that acquires information based on the number of times of simple X-ray imaging as information regarding a continuous imaging dose that is a dose in a series of X-ray imaging processes, and a determiner (controller 211) that determines imaging conditions in the series of X-ray imaging in accordance with the information based on the number of times of simple X-ray imaging acquired by the acquirer Thus, imaging conditions can be determined so that the dose in a series of X-ray imaging operations becomes an appropriate value, based on information (number-of-times information) based on the number of times of simple X-ray imaging. That is, the dose in a series of X-ray imaging operations can be controlled to be an appropriate value.

In the still image capturing, one radiation image is generated in one capturing. Therefore, even if the imaging conditions are inappropriate, the influence on the exposure dose is slight. However, in the dynamic imaging, a plurality of frame images are generated in one imaging. Therefore, in the dynamic imaging, an error from an appropriate imaging condition is accumulated for a plurality of frame images even in one imaging. Thus, in the dynamic imaging, the difference in the setting of the imaging condition has a larger influence as the exposure amount than in the still image imaging.

Further, in dynamic imaging, if imaging is not performed under appropriate imaging conditions, and re-imaging is performed upon determining that an imaging failure has occurred, the amount of exposure becomes enormous compared to still image imaging. Therefore, it is important to set appropriate imaging conditions before imaging.

The imaging control apparatus 21 according to the present embodiment includes an acquirer (controller 211) that acquires information based on the number of times of simple X-ray imaging as information regarding a continuous imaging dose that is a dose in a series of X-ray imaging processes, and a presenter (controller 211) that presents imaging conditions in the series of X-ray imaging processes in accordance with the information based on the number of times of simple X-ray imaging acquired by the acquirer.

Thus, imaging conditions can be presented so that the dose in a series of X-ray imaging operations becomes an appropriate value, based on information (number-of-times information) based on the number of times of simple X-ray imaging. That is, the dose in a series of X-ray imaging operations can be controlled to be an appropriate value.

According to the imaging control apparatus 21 of the present embodiment, the imaging conditions in the series of X-ray imaging are conditions corresponding to the subject.

Thus, it is possible to determine and present imaging conditions according to the subject such that the dose in a series of X-ray imaging operations becomes an appropriate value.

The imaging control apparatus 21 according to the present embodiment includes an acquirer (controller 211) that acquires information based on the number of times of a first simple X-ray imaging as information regarding a continuous imaging dose that is a dose in a series of X-ray imaging processes, an acceptor (controller 211) that accepts input of the imaging condition n the series of X-ray imaging, and an alert outputter (controller 211) that outputs an alert based on the information based on the number of times of first simple X-ray imaging acquired by the acquirer and the imaging conditions in the series of X-ray imaging processes accepted by the acceptor.

Thus, if the imaging conditions m the series of X-ray imaging accepted by the acceptor are not conditions such that the dose has an appropriate value, the user can be notified of the above. That is, the dose in a series of X-ray imaging operations can be controlled to be an appropriate value.

In the imaging control apparatus 21 according to the present embodiment, the alert outputter (controller 211) outputs an alert when the information based on the number of times of the second simple X-ray imaging under the imaging conditions in the series of X-ray imaging accepted by the acceptor (controller 211) exceeds the information based on the number of times of the first simple X-ray imaging.

Thus, if the number-of-tunes information based on the imaging conditions in the series of X-ray imaging accepted by the acceptor exceeds the upper limit value of the allowable range, the user can be notified of the above.

The imaging control apparatus 21 of the present embodiment includes the dose acquirer (controller 211) that acquires the dose of radiation emitted to the subject, and the alert outputter (contoller 211) outputs the alert when the information based on the number of times of the third simple X-ray imaging based on the dose of radiation emitted to the subject acquired by the dose acquirer exceeds a value smaller than the information based on the number of times of the first simple X-ray imaging by a predetermined value.

Thus, before the number-of-times information based on the actual measurement value of the dose of radiation acquired by the dose acquirer exceeds the upper limit value of the allowable range, the user can be notified of the above.

The imaging control apparatus 21 according to the present embodiment includes an acquirer (controller 211) that acquires information based on the number of times of first simple X-ray imaging as information about a continuous imaging dose that is a dose in a series of X-ray imaging, an acceptor (controller 211) that accepts input of imaging conditions in the series of X-ray imaging, and a limiter (controller 211) that limits setting of imaging conditions or limits the series of X-ray imaging, based on the information based on the number of times of first simple X-ray imaging acquired by the acquirer and the imaging conditions in the series of X-ray imaging accepted by the acceptor.

Thus, in a case where the imaging conditions in the series of X-ray imaging accepted by the acceptor are not conditions under which the dose becomes an appropriate value, setting of the imaging conditions can be limited. Alternatively, in this case, a series of X-ray imaging operations can be limited. That is, the dose in a series of X-ray imaging operations can be controlled to be an appropriate value.

In the imaging control apparatus 21 according to the present embodiment, the limiter (controller 211) limits the setting of the imaging condition or limits the string of X-ray imaging when the information based on the number of times of the second simple X-ray imaging under the imaging conditions in the series of X-ray imaging accepted by the acceptor (controller 211) exceeds the information based on the number of times of the first simple X-ray imaging.

Thus, if the number-of-times information based on the imaging conditions in the series of X-ray imaging accepted by the acceptor exceeds the upper limit value of the allowable range, the setting of the imaging condition can be limited. Alternatively, in this case, a series of X-ray imaging operations can be limited.

The imaging control apparatus 21 of the present embodiment includes the dose acquirer (controller 211) that acquires the dose of radiation emitted to the subject, and the controller 211 that stops the emission of radiation when the information based on the number of times of the third simple X-ray imaging based on the dose of radiation emitted to the subject acquired by the dose acquirer exceeds a value smaller than the information based on the number of times of the first simple X-ray imaging by a predetermined value.

Thus, before the number-of-times information based on the actual measurement value of the dose of radiation acquired by the dose acquirer exceeds the upper limit value of the allowable range, the emission of radiation can be stopped.

According to the imaging control apparatus 21 of the present embodiment, the information based on the number of times of simple X-ray imaging is information corresponding to at least one of the age of the subject, the sex of the subject, the physique of the subject, the dose in past X-ray imaging of the subject, and a standard dose.

Thus, the dose in the series of X-ray imaging can be controlled to be an appropriate value based on the information (number-of-times information) based on the number of times of simple X-ray imaging corresponding to at least one of the age of the subject, the sex of the subject, the physique of the subject, the dose in the past X-ray imaging of the subject, and the standard dose.

In the imaging control apparatus 21 of the present embodiment, the acquirer (controller 211) acquires information based on the number of times of simple X-ray imaging from the radiology department information system.

Thus, information based on the number of times of simple X-ray imaging can be easily acquired.

In the imaging control apparatus 21 of the present embodiment, the imaging conditions are at least one of a tube voltage, a tube current, a frame rate, and an imaging time.

Thus, at least one of the tube voltage, tube current, frame rate, and imaging time can be set so that the dose in a series of X-ray imaging operations becomes an appropriate value.

Although the present invention has been described based on the above embodiments, the description in the above embodiments is of preferred examples of the radiographic imaging system according to the present invention, and the present invention is not limited thereto.

For example, the imaging conditions in the series of X-ray imaging operations corresponding to the information based on the number of simple X-ray imaging operations may not be conditions corresponding to the subject as long as the imaging conditions are conditions determined to be appropriate by a doctor or the like.

Further, in the above description, an example in which a hard disk, a semiconductor nonvolatile memory, or the like is used as a computer-readable medium of the program according to the present invention has been disclosed, but the present invention is not limited to this example. Other applicable computer-readable media include portable recording media such as CD-ROM. In addition, a carrier wave is also applied as a medium for providing data of the program according to the present invention via a communication line.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-092262 filed on Jun. 5, 2023, including description, claims, drawings and abstract is incorporated herein by reference.

What is claimed is:

1. An imaging control apparatus comprising:
a hardware processor that acquires information based on a number of times of simple X-ray imaging operations as information regarding a continuous imaging dose which is a dose in a series of X-ray imaging operations,
wherein the hardware processor determines an imaging condition in the series of X-ray imaging operations in accordance with the acquired information based on the number of times of the simple X-ray imaging operations.

2. An imaging control apparatus comprising:
a hardware processor that acquires information based on a number of times of simple X-ray imaging operations as information regarding a continuous imaging dose which is a dose in a series of X-ray imaging operations,
wherein the hardware processor presents an imaging condition in the series of X-ray imaging operations in accordance with the acquired information based on the number of times of the simple X-ray imaging operations.

3. The imaging control apparatus according to claim 1, wherein the imaging condition m the series of X-ray imaging operations is a condition corresponding to a subject.

4. An imaging control apparatus comprising:
a hardware processor that acquires information based on a number of times of first simple X-ray imaging operations as information regarding a continuous imaging dose which is a dose in a series of X-ray imaging operations,
wherein the hardware processor accepts an input of an imaging condition in the series of X-ray imaging operations, and
wherein the hardware processor (i) outputs an alert based on information according to the acquired number of times of the first simple X-ray imaging operations and based on the accepted imaging conditions in the series of X-ray imaging operations or (ii) limits setting of the imaging condition or limits the series of X-ray imaging operations.

5. The imaging control apparatus according to claim 4, wherein the hardware processor outputs an alert when information based on the number of times of second simple X-ray imaging operations based on the accepted imaging conditions in the series of X-ray imaging operations exceeds information based on the number of times of the first simple X-ray imaging operations.

6. The imaging control apparatus according to claim 4, wherein the hardware processor acquires a dose of radiation emitted to a subject, and outputs an alert when information based on a number of times of third simple X-ray imaging operations based on the acquired dose of radiation emitted to the subject exceeds a value smaller than information based on the number of times of the first simple X-ray imaging operations by a predetermined value.

7. The imaging control apparatus according to claim 4, wherein when information based on a number of times of second simple X-ray imaging operations based on the accepted imaging condition in the series of X-ray imaging operations exceeds information based on the number of times of the first simple X-ray imaging operations, the hardware processor limits setting of the imaging condition or limits the series of X-ray imaging operations.

8. The imaging control apparatus according to claim 4, wherein the hardware processor acquires a dose of radiation emitted to a subject, and stops emission of the radiation when information based on a number of times of third simple X-ray imaging operations based on the dose of radiation emitted to the subject exceeds a value smaller than information based on the number of times of the first simple X-ray imaging operations by a predetermined value.

9. The imaging control apparatus according to claim 1, wherein the information based on the number of times of simple X-ray imaging operations is information corresponding to at least one of an age of a subject, a sex of the subject, a physique of the subject, a dose in past X-ray imaging operations of the subject, and a standard dose.

10. The imaging control apparatus according to claim 1, wherein the hardware processor acquires information based on the number of times of the simple X-ray imaging operations from a radiology department information system.

11. The imaging control apparatus according to claim 1, wherein the imaging condition is at least one of a tube voltage, a tube current, a frame rate, and an imaging time.

12. An imaging control method comprising:
acquiring information based on a number of times of simple X-ray imaging operations as information regarding a continuous imaging dose which is a dose in a series of X-ray imaging operations,
wherein an imaging condition in the series of X-ray imaging operations is determined in accordance with the acquired information based on the number of times of the simple X-ray imaging operations.

13. An imaging control method comprising:
acquiring information based on a number of times of simple X-ray imaging operations as information regarding a continuous imaging dose which is a dose in a series of X-ray imaging operations,
wherein an imaging condition in the series of X-ray imaging operations is determined in accordance with the acquired information based on the number of times of the simple X-ray imaging operations.

14. An imaging control method comprising:
acquiring information based on a number of times of simple X-ray imaging operations as information regarding a continuous imaging dose which is a dose in a series of X-ray imaging operations,
accepting an input of an imaging condition in the series of X-ray imaging operations, and
(i) outputting an alert based on the acquired information according to the number of times of the simple X-ray imaging operations and based on the accepted imaging condition in the series of X-ray imaging operations or (ii) limiting setting of the imaging condition or limiting the series of X-ray imaging operations.

15. A non-transitory recording medium including a program that controls a computer of an imaging control apparatus to function as, a hardware processor that performs the method according to claim 12.

16. A non-transitory recording medium including a program that controls a computer of an imaging control apparatus to function as, a hardware processor that performs the method according to claim 13.

17. A non-transitory recording medium including a program that controls a computer of an imaging control apparatus to function as, a hardware processor that performs the method according to claim 14.
